SEQUENCE LISTING (10 sequences)

SEQ ID NO: 1 - RNA, 22 nt, Homo sapiens
ugagguaguagguuguauaguu

SEQ ID NO: 2 - RNA, 21 nt, Homo sapiens
ugagguaggagguuguauagu

SEQ ID NO: 3 - RNA, 24 nt, Homo sapiens
aaaagugcuuacagugcagguagc

SEQ ID NO: 4 - RNA, 21 nt, Homo sapiens
cauuauuacuuuugguacgcg

SEQ ID NO: 5 - RNA, 23 nt, Homo sapiens
uauggcuuuuuauuccuauguga

SEQ ID NO: 6 - RNA, 17 nt, Homo sapiens
agcugguguugugaauc

SEQ ID NO: 7 - RNA, 22 nt, Homo sapiens
uagguuauccguguugccuucg

SEQ ID NO: 8 - RNA, 22 nt, Homo sapiens
aaucauacacgguugaccuauu

SEQ ID NO: 9 - DNA, 45 bp, Artificial
  Synthetic
taatacgactcactataggggattcacaacaccagcttcttcaca

SEQ ID NO: 10 - DNA, 48 bp, Artificial
  Synthetic
taatacgactcactataggggctacctgcactgtaagcactttttctt

(19) United States
(12) Patent Application Publication
(10) Pub. No.: US 2009/0023151 A1
(43) Pub. Date: Jan. 22, 2009

(54) Method For The Labeling And Detection Of Small Polynucleotides

(76) Inventors: Elliott P. Dawson, Murfreesboro, TN (US); Kristie E. Womble, Franklin, TN (US)

Correspondence Address: SHELDON MAK ROSE & ANDERSON PC, 100 Corson Street, Third Floor, PASADENA, CA, 91103-3842, US

(21) Appl. No.: 12/202,133

(22) Filed: Aug. 29, 2008

Related U.S. Application Data
(63) Continuation-in-part of application No. 11/958,180, filed on Dec. 17, 2007.
Application No. 11/958,180 is a continuation-in-part of application No. PCT/US07/77311, filed on Aug. 30, 2007.
(60) Provisional application No. 60/973,098, filed on Sep. 17, 2007.
(60) Provisional application No. 60/824,068, filed on Aug. 30, 2006.
(60) Provisional application No. 60/825,888, filed on Sep. 15, 2006.
(60) Provisional application No. 60/863,886, filed on Nov. 1, 2006.
(60) Provisional application No. 60/866,210, filed on Nov. 16, 2006.
(60) Provisional application No. 60/871,094, filed on Dec. 20, 2006.

(51) Int. Cl. C12Q 1/68; C12P 19/34

(52) U.S. Cl. 435/6; 435/91.5; 435/91.2

(57) ABSTRACT

Methods and kits for use in isolating, labeling or detecting a small polynucleotide of interest from a sample. The method entails hybridizing the small polynucleotide to a capture probe, lengthening the small polynucleotide by primer extension and/or ligation, and degrading the capture probe to provide a single stranded extension product. The kits include the capture probe and additional reagents for the extension, ligation and/or degradation reactions.

### CROSS-REFERENCE TO RELATED APPLICATIONS

The present application is a continuation-in-part of U.S. patent application Ser. No. 11/958,180, “Method and Substances for Isolating RNAs,” filed Dec. 17, 2007, and claims the benefit of U.S. Provisional Patent Application No. 60/973,098, titled “Method for the Labeling and Detection of Small Polynucleotides” filed Sep. 17, 2007. U.S. patent application Ser. No. 11/958,180 is a continuation-in-part of PCT Patent Application PCT/US07/77311, titled “Method and Substances for Isolating RNAs,” filed Aug. 30, 2007, which claims the benefit of U.S. Provisional Patent Application 60/824,068, titled “Method and Substances for Isolating RNAs,” filed Aug. 30, 2006; and claims the benefit of U.S. Provisional Patent Application 60/825,888, titled “Method and Substances for Isolating RNAs,” filed Sep. 15, 2006; and claims the benefit of U.S. Provisional Patent Application 60/863,886, titled “Method and Substances for Isolating RNAs,” filed Nov. 1, 2006; and claims the benefit of U.S. Provisional Patent Application 60/866,210, titled “Method and Substances for Isolating RNAs,” filed Nov. 16, 2006; and claims the benefit of U.S. Provisional Patent Application 60/871,094, titled “Method and Substances for Isolating RNAs,” filed Dec. 20, 2006; the contents of which are incorporated in this disclosure by reference in their entirety.

### BACKGROUND

There are a large variety of small polynucleotides, both naturally occurring and synthetic, which are of scientific or commercial interest. Exemplary small polynucleotides include microRNAs, snoRNAs, short interfering RNAs (natural or synthetic), guide RNAs, nucleolar RNAs, ribosomal RNAs, tRNAs as well as small antisense DNAs or small polynucleotide degradation products. Of particular interest are microRNAs (miRNAs), naturally occurring, single stranded polyribonucleotides (polyRNAs) of between 18 and 24 RNA residues, which are derived from a longer, naturally occurring noncoding eukaryotic precursor RNA transcript (usually having a ‘hairpin’ configuration), and miRNAs play a significant role in cellular developmental and differentiation pathways. Consequently, there have been considerable efforts made to understand and characterize the temporal, spatial and cellular expression levels and patterns of expression of miRNAs to ascertain their precise role in cellular development and differentiation in both normal and disease states.

miRNAs are currently studied by, first, obtaining the total RNA from a sample. Next, the total RNA is fractionated into subpopulations by gel electrophoresis or by chromatographic fractionation and size selective elution. Then, the appropriate section of the gel is cut, and the 18-24 RNAs are eluted from the gel, or the eluted fraction containing single stranded RNA's in the size range of 18-24 ribonucleotides is collected, usually as the RNA fraction of less than 500-200 nucleotides in length. Next, the RNAs are isolated by precipitation and the miRNAs are characterized.

However, these methods are disadvantageous because they do not work well when the amount of sample is small, such as samples from tumor tissue or biopsy material. Further, characterization of the miRNAs isolated by present methods usually comprises a several step amplification procedure followed by detection, quantitation, cloning and sequencing. Because of the large number of steps in these processes, and the notorious inefficiencies associated with the repeated purification, isolation and identification of miRNAs, it is time consuming, relatively expensive, requires relatively large amounts of material and is not fully representative of the population of miRNAs expressed within a sample, such as within a tumor, or of those miRNAs expressed in low abundance. Additionally, the methods are not specific to isolating and identifying miRNAs, and often isolate and identify siRNA, tRNA, 5S/5.8SrRNA and degraded RNA from additional cellular RNAs.

Therefore, there is the need for an improved method for isolation and identification of miRNAs, other small regulatory RNAs and short interfering RNAs (siRNAs) that is not associated with these disadvantages.

### SUMMARY

According to one embodiment of the invention, there is provided a method for labeling and/or detecting small polynucleotides using a capture probe. The capture probe is a polynucleotide that includes (1) a small polynucleotide binding segment having a small polynucleotide binding segment sequence, the small polynucleotide binding segment having a 3′ end and a 5′ end; (2) a template segment having a template segment sequence, the template segment having a 3′ end and a 5′ end; and, optionally, (3) a spacer segment having a spacer segment sequence, the spacer segment having a 3′ end and a 5′ end. The 3′ end of template segment is connected to the 5′ end of the small polynucleotide binding segment and, when present, the 5′ end of the spacer segment is connected to the 3′ end of the small polynucleotide binding segment.

The small polynucleotide binding segment is substantially complementary to, and capable of hybridizing to, one or more than one small polynucleotides of interest by Watson-Crick base pairing. In preferred versions of the capture probe the small polynucleotide of interest is selected from the group consisting of miRNA, snoRNA, siRNA and short interfering RNA.

The method includes the steps of (a) providing one or more than one capture probe as set forth above; (b) providing a sample comprising a small polynucleotide of interest; (c) combining the capture probe and the sample; (d) allowing the small polynucleotide of interest to hybridize with the small polynucleotide binding segment of the capture probe to form a small polynucleotide/capture probe complex; (e) combining the small polynucleotide/capture probe complex with a polynucleotide polymerase, preferably a polymerase capable of using RNA as a primer, and a set of nucleoside triphosphates; (f) extending the hybridized small polynucleotide of interest to form an extension product, where the extension product comprises the small polynucleotide of interest connected at the 3′ end to an extended segment, the extended segment sequence comprising a sequence complementary to the template segment of the capture probe, and where the extension product is hybridized to the capture probe to form an extension product/capture probe complex; and (g) degrading the capture probe but not the extension product to obtain a single stranded extension product.

In preferred versions of the method the small polynucleotide of interest is selected from the group consisting of miRNAs, snoRNAs, siRNAs or short interfering RNAs. In a particularly preferred version, the small polynucleotide of interest is a miRNA.

In one embodiment of the method, the capture probe also contains a solid phase binding segment and the small polynucleotide/capture probe complex or the extension product/capture probe complex is captured to a solid phase by binding of capture probe to a solid support via the solid phase binding segment prior to degradation of the capture probe and release of the single stranded extension product.

In another embodiment of the method, the capture probe further includes a spacer segment having a spacer segment sequence, the spacer segment having a 3′ end and a 5′ end, where the 5′ end of the spacer segment is connected to the 3′ end of the small polynucleotide binding segment.

In one embodiment of the method the one or more than one capture probe is a composition comprising two or more capture probes. The composition includes: (a) a first capture probe having a first small polynucleotide binding segment and a first template segment; and (b) a second capture probe having a second small polynucleotide binding segment and a second template segment, where the second small polynucleotide binding segment has a different polynucleotide binding segment sequence than the first polynucleotide binding segment and the second template segment has a different template segment sequence than the first template segment.

In a preferred embodiment of the method one or more than one of the nucleoside triphosphates contains a detectable label. In another preferred embodiment, at least three of the nucleoside triphosphates are nucleoside triphosphate analogs, where the alpha-phosphorus atom of the nucleoside triphosphate is replaced by sulfur. In a particularly preferred embodiment at least three of the nucleotide triphosphates are selected from the group consisting of 2′-Deoxyadenosine-5′-O-(1-Thiotriphosphate), 2′-Deoxyguanosine-5′-O-(1-Thiotriphosphate), 2′-Deoxycytidine-5′-O-(1-Thiotriphosphate), and 2′-Deoxythymidine-5′-O-(1-Thiotriphosphate).

In one embodiment of the method the extension product is a chimeric polynucleotide, where the polynucleotide of interest is a small RNA and the extended segment is a DNA polynucleotide.

In a preferred embodiment of the method the extension product is a chimeric polynucleotide, where the polynucleotide of interest is a small RNA the extended segment contains a phosphorothioate backbone. The degrading step can then include treating the extension product/capture probe complex with one or more than one nuclease. In a particularly preferred embodiment the nuclease is DNase I.

In an alternative embodiment of the method, the capture probe is a phosphorothiolated polynucleotide and the degrading step comprises treating the extension product/capture probe complex with iodine.

One embodiment provides another method for labeling and/or detecting a small RNA of interest in a sample, which includes the steps of: (a) extending the small polynucleotide of interest as set forth above; (b) providing a ligase and a linker segment, the linker segment comprising a polynucleotide having 3′ end and a 5′ end, the linker segment having a linker segment sequence, wherein the linker segment sequence is substantially complementary to, and capable of hybridizing to, the spacer segment sequence by Watson-Crick base pairing; (c) allowing the linker segment to hybridize to the spacer segment; (d) ligating the 3′ end of the linker segment to the 5′ end of the small RNA of interest to form a ligated extension product substantially complementary to, and capable of hybridizing to, the capture probe sequence; and (d) degrading the capture probe to obtain a single stranded ligated extension product.

In one embodiment of this method, the one or more than one capture probe is a composition comprising two or more capture probes. The composition includes: (a) a first capture probe having a first spacer segment, a first small polynucleotide binding segment and a first template segment; and (b) a second capture probe having a second spacer segment, a second small polynucleotide binding segment and a second template segment, where the second small polynucleotide binding segment has a different polynucleotide binding segment sequence than the first polynucleotide binding segment and the second template segment has a different template segment sequence than the first template segment.

In another embodiment of this method the ligated extension product is a chimeric polynucleotide, wherein polynucleotide of interest is a small RNA and both the linker segment and the extended segment are DNA polynucleotides.

In yet another embodiment of this method the ligated extension product is a chimeric polynucleotide, where polynucleotide of interest is a small RNA, and the linker segment and the extended segment both contain a phosphorothioate backbone. The degrading step of the method can then include treating the ligated extension product/capture probe complex with one or more than one nuclease. In a preferred embodiment, the nuclease is DNase I.

In an alternative embodiment of the method, the capture probe is a phosphorothiolated polynucleotide and the degrading step comprises treating the ligated extension product/capture probe complex with iodine.

In one version of the method, the linker segment or the extended segment contains a detectable label. An alternative version of the method further comprises amplifying the ligated extension product by a polymerase chain reaction.

One embodiment provides a kit for the isolation, labeling and detection of small RNAs, which can include one or more capture probes as described above, and one or more additional reagents for carrying out various steps of the methods, such as (1) a deoxynucleoside triphosphate mix comprising the alpha thio triphosphate forms of dATP, dCTP, and dGTP and a labeled form of dUTP; (2) a polymerase capable of extending the 3′ end of a polynucleotide of interest hybridized to the capture probe using the extension template segment of the capture probe as a template for an extension reaction; (3) an oligonucleotide linker that is substantially complementary to and capable of hybridizing to the spacer segment of the capture probe; (4) a ligase enzyme; (5) DNase I; (6) alkaline phosphatase; (7) suitable buffers compatible with one or more of the polymerase, ligase, DNase I and alkaline phosphatase reactions; (8) spin columns for separation of unincorporated nucleoside triphosphates from labeled extension products; and (9) streptavidin coated paramagnetic beads. The invention is described in more detail by the following description.

### FIGURES

These and other features, aspects and advantages of the present invention will become better understood with regard to the following description, appended claims, and accompanying figures where:

FIG. 1 is a schematic diagram of some of the steps in certain embodiments of a method of labeling and/or detecting miRNAs and other small polynucleotides using a capture probe according to the present invention;

FIG. 2 shows diagrams of some of the steps in certain other embodiments of a method for labeling and/or detecting miRNAs or other small polynucleotides using a capture probe to guide ligation of a linker according to the present invention;

FIG. 3 shows electropherograms analyzing the end products of some of the steps in one embodiment of a method for labeling and detecting miRNAs using a capture probe to guide an extension reaction labeling a miRNA of interest.

### DESCRIPTION

According to one embodiment of the present invention, there is provided a method for labeling and/or detecting small polynucleotides, such as for example miRNAs (small RNAs), short interfering RNAs and other small regulatory RNAs and DNAs. According to another embodiment of the present invention, there is provided a method for labeling small polynucleotides of interest. In one embodiment, the method for detecting small polynucleotides of interest comprises, first, labeling the small polynucleotides of interest according to the present invention. According to another embodiment of the present invention, there is provided a method utilizing one or more than one capture probe or one or more than one set of capture probes for labeling or detecting small polynucleotides via an extension reaction. In one embodiment, the method for labeling or detection small polynucleotides further includes a ligation reaction. The method and capture probes will now be disclosed in detail.

As used in this disclosure, except where the context requires otherwise, the term “comprise” and variations of the term, such as “comprising,” “comprises” and “comprised” are not intended to exclude other additives, components, integers or steps.

As used in this disclosure, the term “small RNAs” means a naturally occurring, single stranded RNA of between 18 and 24 RNA residues, usually with a 5′ terminal phosphate group, usually referred to as “mature micro RNAs,” which is derived from a larger naturally occurring precursor RNA, usually having a “hairpin” configuration.

As used in this disclosure the terms “small polynucleotide” and “small polynucleotides” refer to polynucleotides which are between 17 and 200 residues in length, usually single stranded RNA or DNA, which encompasses the group of noncoding regulatory RNAs including for example miRNAs, snoRNAs, snRNAs, siRNAs, antisense DNAs and Okazaki fragments.

As used in this disclosure, the terms “one or more than one small polynucleotide,” “a small polynucleotide” and “the small polynucleotide” are intended to be synonymous, that is are intended to indicate either one small polynucleotide of interest or a plurality of small polynucleotides of interest, except where the context requires otherwise.

As used in this disclosure, the terms “one or more than one capture probe,” “a capture probe,” “the capture probe,” “the capture probes,” “capture-extension probe,” “capture-extension probes,” “capture and extension template probe” and “capture and extension template probes” are intended to be synonymous, and are intended to indicate either the singular or plural, except where the context requires otherwise.

As used in this disclosure, the term “substantially complementary” and variations of the term, such as “substantial complement,” means that at least 90% of all of the consecutive residues in a first strand are complementary to a series of consecutive residues of the same length of a second strand. As will be understood by those with skill in the art with reference to this disclosure, one strand can be shorter than the other strand and still be substantially complementary. With respect to the invention disclosed in this disclosure, for example, the small RNA or small RNA binding segment can be shorter or longer than the complementary small RNA of interest; however, it is preferable that the small RNA binding segment is of the same length and is substantially complementary to its corresponding small RNA.

As used in this disclosure, the term “hybridize” and variations of the term, such as “hybridizes” and “hybridized,” means a Watson-Crick base pairing of complementary nucleic acid single strands or segments of strands to produce an anti-parallel, double-stranded nucleic acid, and as used in this disclosure, hybridization should be understood to be between substantially complementary strands unless specified otherwise, or where the context requires otherwise. As an example, hybridization can be accomplished by combining equal molar concentrations of each of the pairs of single strands, such as 100 pmoles, in the presence of 5 ug yeast tRNA in a total volume of 50 μl of aqueous buffer containing 400 mM MOPS, 80 mM DTT, and 40 mM MgCl₂ at a pH of 7.3, and then incubating the mixture at 25° C. for one hour while shaking gently. Additionally buffers other than MOPS and blocking agents other than tRNA salts other than magnesium and pH ranges of from 5-9 are generally utilized and are well known in the art. However, the range of pH usually used for hybridization is on the order of 6-9.

As used in this disclosure, the term “near the end” and variations of the term, means within 20% of the residues of the identified end residue. For example, near the end of a 20 residue strand, means the first four residues of the identified 5′ or 3′ end or terminus end of the strand.

As used in this disclosure, the terms “extension” or “extension reaction” indicates the extension of the 3′ end of a polynucleotide by the action of a polymerase in conjunction with all the accessory reagents and conditions for this reaction to occur.

### Methods of Use

According to one embodiment of the present invention, there is provided a method for labeling an miRNA (microRNA) or other small polynucleotides of interest from a sample comprising the small polynucleotide of interest. According to another embodiment of the present invention, there is provided a method for identifying miRNAs or other small polynucleotides. In one embodiment, the method for identifying miRNAs or other small polynucleotides comprises, first, labeling the small polynucleotides according to the present invention.

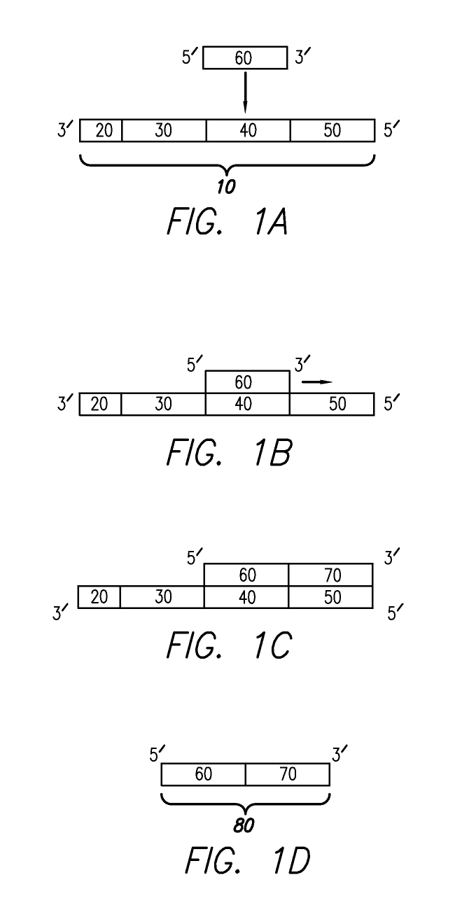

Referring now to FIG. 1, there are shown some of the steps in certain embodiments of the methods. The steps shown are not intended to be limiting nor are they intended to indicate that each step depicted is essential to the method, but instead are exemplary steps only.

Capture Probes

As can be seen in FIG. 1A, the method comprises, first, providing a capture probe 10. According to one embodiment of the present invention, there is provided a capture probe 10 suitable for use with a method for isolating small RNAs or DNAs. Referring to FIG. 1A, the capture probe comprises from its 3′ end to its 5′ end covalently joined or connected segments: a) an optional solid phase binding segment 20, b) an optional spacer segment 30, c) a small polynucleotide binding segment 40 having a small polynucleotide binding segment sequence, where the small polynucleotide binding segment is substantially complementary to and capable of hybridizing to one or more than one small polynucleotide of interest by Watson-Crick base pairing, and d) a template segment 50.

In one embodiment, the capture probe 10 comprises a substance selected from the group consisting of one or more than one type of polynucleotide, one or more than one of polynucleotide analog, and a combination of one or more than one type of polynucleotide and polynucleotide analog.

In one embodiment the capture probe comprises a solid phase binding segment 20 of a molecular composition capable of binding to a solid phase, such as for example biotin coupled to the 3′ end of the capture probe and its ability for binding to avidin or streptavidin immobilized to a solid phase, such as for example streptavidin coated paramagnetic particles or streptavidin coated wells of a microtiter plate. In another embodiment, the solid phase binding segment 20 is a substance capable of covalent binding to a solid phase, such as for example a primary amine coupled to carboxylic acid groups on a solid phase using carbodiimide activation and amide bond formation in between the primary amine of the solid phase binding segment and the carboxylic acid groups on the solid phase. Other suitable methods of covalent coupling of polynucleotides to solid phases are well known in the art. In one embodiment, the solid phase binding segment 20 is either the 3′, 5′ or both ends of the capture probes 10, they may also be interior to either the spacer segment 30 or the template segment 50 or both segments of the capture probes 10. Further the solid phase binding segment 20 can be added during the synthesis of the capture-extension probes 10, for example as a biotin phosphoramidite during polynucleotide synthesis as will be understood by those skilled in the art. In addition the solid phase binding segment 20 can be introduced after the synthesis of the a contiguous capture probe containing the spacer segment 30, the small polynucleotide binding segment 40 and the template segment 50, for example by the incorporation of a biotin labeled dUTP to the 3′ terminus of the capture probe by the action of terminal transferase using biotinylated dUTP as the source for biotin.

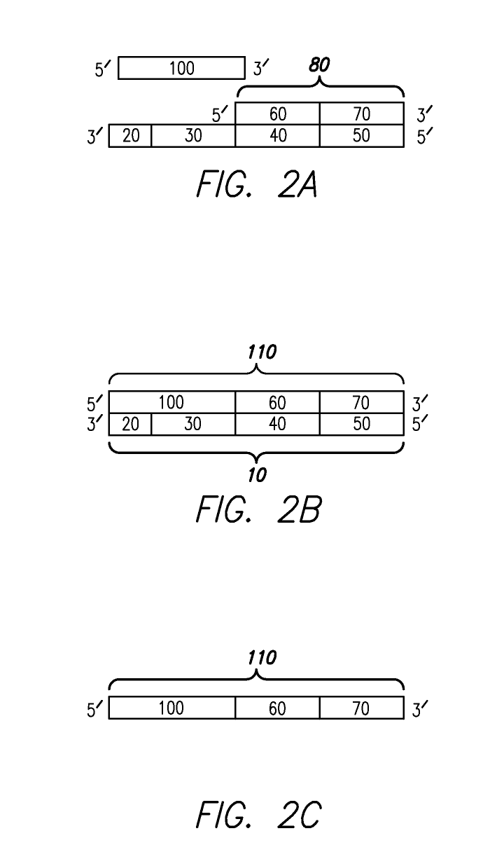

In one embodiment, the spacer segment 30 of the capture probe comprises a polynucleotide sequence, having a predetermined sequence or predetermined size. In one embodiment, the spacer segment is of sufficient length to minimize steric hindrance of hybridization complexes forming with the polynucleotide binding segment. In one embodiment the spacer segment includes a primer binding site for subsequent amplification reactions. In another embodiment, shown in FIG. 2, the spacer segment includes a docking site for a linker in ligation reactions. In yet another embodiment, the spacer includes one or more than one desired restriction enzyme recognition site.

The polynucleotides of the spacer segment 30 may be naturally occurring, synthetic or nucleotide analogs comprising 5-50 nucleotides, or 5-40 nucleotides, preferably 5-30 nucleotides. In one embodiment, the spacer segment 30 consists of RNA. In one embodiment, the spacer segment 30 consists of DNA. In one embodiment, the spacer segment 30 consists of polynucleotide analogs. In one embodiment, the spacer segment 30 consists of a chimera of more than one polynucleotide or polynucleotide analog selected from the group consisting of RNA, DNA, polynucleotide analogs of RNA, and polynucleotide analogs of DNA.

The small polynucleotide binding segment 40 is designed to form a hybridization complex with a polynucleotide of interest. In one embodiment, the small polynucleotide of interest is a small RNA molecule. In one embodiment, the small polynucleotide of interest is a small DNA molecule. In one embodiment, the small polynucleotide binding segment 40 consists of between 18 and 24 DNA residues. In another embodiment, the small polynucleotide binding segment 40 consists of 18 or 19 or 20 or 21 or 22 or 23 or 24 DNA residues. In another embodiment the small polynucleotide binding segment 40 comprises a DNA of between 17 and 100 polynucleotides. In another embodiment the small polynucleotide binding segment 40 comprises a DNA of between 17 and 60 polynucleotides. In another embodiment the small polynucleotide binding segment 40 comprises between 17 and 40 polynucleotides.

The small polynucleotide binding segment 40 is substantially complementary to, and capable of hybridizing to, one or more than one small polynucleotide of interest by Watson-Crick base pairing, including a small polynucleotide of interest having a predetermined sequence or having a predetermined size, from a sample comprising substances that are chemically related, such as for example, a mixture of messenger RNAs, transfer RNAs, ribosomal RNAs and genomic DNA. A small polynucleotide of interest 60 can be selected from any known small RNA from any suitable source, as will be understood by those with skill in the art with reference to this disclosure. In one embodiment, the small polynucleotide of interest 60 is selected from a public database. In a preferred embodiment, the small polynucleotide of interest 60 is an miRNA and the public database is a central repository provided by the Sanger Institute http://miRNA.sanger.ac.uk/sequences/ to which newly discovered and previously known miRNA sequences can be submitted for naming and nomenclature assignment, as well as placement of the sequences in a database for archiving and for online retrieval via the world wide web. Generally, the data collected on the sequences of miRNAs by the Sanger Institute include species, source, corresponding genomic sequences and genomic location (usually chromosomal coordinates), as well as full length transcription products and sequences for the mature fully processed miRNA.

To select the sequence or sequences of the small polynucleotide binding segment 40, when the target small RNAs comprise miRNA, a miRNA of interest or set of miRNAs of interest are selected from a suitable source, such as for example, the Sanger Institute database or other suitable database, as will be understood by those with skill in the art with reference to this disclosure. If a set of miRNAs of interest is selected from a source that contains duplicate entries for one or more than one miRNAs, in a preferred embodiment, the duplicated entries are first removed so that the set of sequences of miRNAs of interest contains only one sequence for each miRNA of interest. In one embodiment, the set of miRNAs of interest consists of one of each miRNAs from a single source or database, such as one of each miRNAs listed in the central repository provided by the Sanger Institute.

In another embodiment the small polynucleotide of interest 60 is a eucaryotic small RNA. In another embodiment the small RNA of interest is a primate small RNA. In another embodiment the small RNA of interest is a virus small RNA. In a preferred embodiment, the small RNA of interest is a human small RNA. In another embodiment, the set of small RNAs of interest are all eucaryotic miRNAs. In another embodiment, the set of small RNAs of interest are all primate miRNAs. In another embodiment, the set of small RNAs of interest are all human miRNAs.

In another embodiment the small polynucleotide of interest 60 is a eucaryotic small DNA. In another embodiment the small DNA of interest is a primate small DNA. In another embodiment the small DNA of interest is a virus small DNA. In a preferred embodiment, the small DNA of interest is a human small DNA. In another embodiment, the set of small DNAs of interest are all eucaryotic DNAs. In another embodiment, the set of small DNAs of interest are all primate DNAs. In another embodiment, the set of small DNAs of interest are all human DNAs.

In a preferred embodiment, the small polynucleotide binding segment 40 is exactly the complement to the small polynucleotide of interest 60 in both length and sequence. In another embodiment, the small polynucleotide binding segment is a more than 90% complementary to a segment of the small polynucleotide of interest of the same length as the small polynucleotide of interest sequence. In another embodiment, the small polynucleotide binding segment 40 is more than 80% complementary to a segment of the small polynucleotide of interest 60 of the same length as the small polynucleotide of interest sequence 60.

In one embodiment, the small polynucleotide binding segment 40 consists of RNA. In one embodiment, the small polynucleotide binding segment 40 consists of DNA. In one embodiment, the small polynucleotide binding segment 40 consists of polynucleotide analogs. In one embodiment, the small polynucleotide binding segment 40 consists of a chimera of more than one polynucleotide or polynucleotide analog selected from the group consisting of RNA, DNA, polynucleotide analogs of RNA, and polynucleotide analogs of DNA.

Additionally, the small polynucleotide binding segment 40 can be complementary to miRNAs, snoRNAs, siRNAs or short interfering RNAs thereby facilitating their assay.

The template segment 50 of the capture probe comprises a polynucleotide sequence, having a predetermined sequence or predetermined size, designed to provide one or more functional features.

In a particularly preferred embodiment, the polynucleotide comprising the template segment 50 of the capture probe can serve as a template for the synthesis of a complementary polynucleotide strand by the action of a polynucleotide polymerase.

In another embodiment, the template segment comprises one or more than one sequence that is a restriction enzyme recognition motif. In a particularly preferred embodiment, the specific restriction enzyme recognition motif, when present, is not present in the DNA analog of the miRNA or other small polynucleotide of interest that is being isolated and identified by the present methods. In one embodiment, the restriction enzyme recognition motif is recognized by a restriction enzyme selected from the group consisting of BamHI, Hind III and EcoR I. In a preferred embodiment, the restriction site motif is recognized by a restriction enzyme selected from the group consisting of Not I, Xho I, Xma I and Nhe I, because BamH I, Hind III and EcoR I also act upon some DNA equivalents of sequences of miRNA. As will be understood by those with skill in the art with reference to this disclosure, however, other suitable restriction site motifs can also be used.

In one embodiment, the template segment 50 of the capture probe comprises a polynucleotide comprised of nucleotides which are naturally occurring, synthetic or nucleotide analogs.

In one embodiment, the template segment 50 comprises 1-50 nucleotides, in another embodiment the template segment comprises 1-40 nucleotides, and in yet another embodiment the template segment comprises 1-30 nucleotides.

In one embodiment, the template segment 50 consists of RNA. In one embodiment, the template segment 50 consists of DNA. In one embodiment, the template segment 50 consists of polynucleotide analogs. In one embodiment, the template segment consists of a chimera of more than one polynucleotide or polynucleotide analog selected from the group consisting of RNA, DNA, polynucleotide analogs of RNA, and polynucleotide analogs of DNA.

In a preferred embodiment, the capture probe contains one or more than one modified nucleotide or nucleotide analog. For example, the capture probe may contain one or more internucleoside bonds, such as phosphorothioate, boranophosphate, methylphosphonate, or peptide bonds. An alternative example of a nucleotide analog would be where a deoxyuridine is substituted for a deoxythymidine.

In a set of capture probes 10, the template segments 50 can comprise identical sequences, different sequences or different in both sequence and length. For example, template segments 50 comprising polynucleotides of different lengths in a set of capture probes 10, can be used to produce different extension products of their respective target small polynucleotides such as miRNAs. Further, extension products of different lengths can then be utilized to distinguish different target small RNAs from one another using standard methods, such as for example using capillary electrophoresis.

The synthesis of the capture probes 10 entails known methods as will be understood by those with skill in the art with reference to this disclosure. For example, the method can comprise, first, selecting the sequences of solid phase binding segment 20, the spacer segment 30, the small polynucleotide binding segment 40 and the template segment 50, and then synthesizing them. For example, in one embodiment, the 3′ solid phase binding segment 20 comprises biotin, the spacer segment 30 comprises a short DNA polynucleotide segment of 5 nucleotides such as AGCTC, or a polynucleotide such as the T7 DNA dependent RNA promoter or its complementary sequence or other polynucleotide that is not complementary to the small polynucleotide of interest 60, the small polynucleotide binding segment 40 comprises one or more complementary DNA sequence to the small polynucleotide of interest 60, such as the miRNAs listed in Table I of the examples and set forth as SEQ ID NOs 1-8 in the sequence listing, and the template segment 50 comprises a DNA polynucleotide sequence such as for example an SP6 DNA dependent RNA polymerase promoter or other polynucleotide that is not complementary to the small polynucleotide of interest. Additionally, a restriction site can be included in either or both the spacer segment 30 and the template segments 50 of the capture probes.

In a particularly preferred embodiment, the penultimate 3′ end of the capture probe 10 is blocked, for example by phosphate, phosphothioate, biotin, dideoxynucleotide, 3′ amine and the like, so that it cannot be extended. Such blocking of 3′ ends to prevent extension is well known in the art. The purpose of such a blocking terminus is to prevent extension of the capture probe 10 by pseudo or latent terminal transferase activity inherent in several polymerases.

Synthesis of the capture probes 10 can readily be accomplished by phosphoramidite chemistry and can be obtained from a number of sources well known in the art, as will be understood by those with skill in the art with reference to this disclosure. Referring now to Table II of the example, there are shown 2 sample capture probes 10 useful for detecting the small RNAs of interest 60 listed in the left-hand column (both of which are human miRNAs listed in Table I).

In one embodiment, the capture probe 10 provided has the characteristics and attributes as disclosed for a capture probe 10 according to the present invention, some of which will be repeated hereafter for clarity. As can be seen in FIG. 1, the capture probe 10 comprises three segments depicted in FIG. 1 from left to right, from the 3′ end of the capture probe 10 to the 5′ end of the capture probe: a) a spacer segment 30 having a spacer segment sequence; b) a small polynucleotide binding segment 40 having a polynucleotide binding segment sequence; and c) a template segment 50 having a template segment sequence, and comprising a 3′ end and a 5′ end, where the 5′ end of the spacer segment 50 is connected to the 3′ end of the polynucleotide binding segment 40, and where the 5′ end of the polynucleotide binding segment 40 is connected to the 3′ end of the template segment 50. In a preferred embodiment, the specificity of the polynucleotide binding segment 40 to an miRNA or other small polynucleotide of interest 60 allows the method to be used directly on a sample containing substances related to miRNA or on isolated total RNA without requiring the specific separation of miRNAs from the sample or from the total RNA, such as for example by either gel purification or chromatographic purification, as necessary in prior art methods.

In a particularly preferred embodiment, the penultimate 3′ end of the capture probe 10 is blocked, for example by phosphate, phosphothioate, biotin, dideoxynucleotide, 3′ amine and the like, so that it cannot be extended. Such blocking of 3′ ends to prevent extension is well known in the art. The purpose of such a blocking terminus is to prevent extension of the capture probe 10 by pseudo or latent terminal transferase activity inherent in several polymerases.

In one embodiment, a plurality of capture probes 10 are provided as a composition or mixture comprising two or more capture probes. The mixture includes (a) a first capture probe 10 having a first spacer segment 30, a first small polynucleotide binding segment 40 and a first template segment 50; and (b) a second capture probe 10 having a second spacer segment 20, a second small polynucleotide binding segment 40 and a second template segment 50, where the second small polynucleotide binding segment 40 has a different small polynucleotide binding segment sequence than the first small polynucleotide binding segment 40 and the second template segment 50 has a different template segment sequence than the first template segment 50. The presence of different small polynucleotides bound to the capture probes 10 can thus be correlated to a detectable difference in the associated template segments 50. In a preferred embodiment, the first template segment 50 and the second template segment 50 differ in length.

Samples

As can be seen in FIG. 1A, the method further comprises, providing a sample comprising a miRNA or other small polynucleotide of interest 60. Samples suitable for analysis by the present method either comprise or potentially comprise small RNAs and small DNAs. In one embodiment, the sample further comprises one or more than one substance that is chemically related to the miRNA of interest, such as for example, a substance selected from the group consisting of messenger RNA, transfer RNA, ribosomal RNA, siRNA, 5S/5.8SrRNA, genomic DNA and a combination of the preceding. In one embodiment, the sample further comprises one or more than one RNA other than miRNA, such as for example, a substance selected from the group consisting of messenger RNA, transfer RNA, ribosomal RNA, siRNA, 5S/5.8SrRNA and a combination of the preceding. All of the RNA in the sample, regardless of the type of RNA, constitutes the “total RNA” in the sample.

In one embodiment, suitable samples are obtained from eukaryotic cells obtained from whole blood, tissue culture, cell cultures, whole tissues such as liver, lung, brain, or even whole organisms such as C. elegans or Drosophila. Small polynucleotides can also be isolated from tissues infected by some viruses as these microbes produce miRNAs which can suppress the immune response or modify other host factors to enable their persistence and infection by compromising host factors or otherwise divert host resources to their advantage. Also, small polynucleotides can occur in bacteria or procaryotes which regulate their processes such as biofilm formation and other activities of the bacteria such as pathogenicity. Such specimen sources are well known in the art.

In one embodiment, the sample is from a eukaryote. In another embodiment, the sample is from a primate. In a preferred embodiment, the sample is from a human.

In one embodiment, the sample comprises a tissue or fluid selected from the group consisting of blood, brain, heart, intestine, liver, lung, pancreas, muscle, a leaf, a flower, a plant root and a plant stem.

Cell lysates are suitable for use with the capture probes 10, especially when care has been taken to neutralize nucleases which can degrade the miRNAs or small polynucleotides to be examined in the sample or degrade the capture probes 10 contacted with the sample, however, the capture probes can be rendered resistant to the action of nucleases by their synthesis with nuclease resistant backbones such as amides such as peptide nucleic acids or more commonly phosphothioate modified backbones during their synthesis. In another embodiment the sample is a mounted, fixed tissue section, where the fixed small polynucleotides, for example miRNAs, in the sample serve as the solid phase binding segment or element 20 of the capture-extension probes 10.

In one embodiment, the method further comprises isolating the total RNA from the sample after providing the sample. In a preferred embodiment, total RNA is isolated from such specimens using methods well known in the art or using commercial kits widely available from vendors such as QIAgen, Invitrogen, Promega and the like. As will be understood by those with skill in the art with reference to this disclosure, when the method comprises isolating the total RNA from the sample after providing the sample, the term “sample” means the isolated total RNA for the remaining steps in the method.

The small polynucleotide of interest 60 has a small polynucleotide of interest sequence, and comprises 3′ end and a 5′ end. In one embodiment, the small polynucleotide of interest is a miRNA, which consists of between 18 and 24 RNA residues. In another embodiment, the miRNA of interest consists of 18 or 19 or 20 or 21 or 22 or 23 or 24 RNA residues.

The small polynucleotide of interest 60 is substantially complementary to, and capable of hybridizing to, a small polynucleotide binding segment 40 of a capture probe 10 according to the present invention by Watson-Crick base pairing. In one embodiment, the small polynucleotide is a miRNA of interest listed in a public database. In a preferred embodiment, the public database is a central repository provided by the Sanger Institute http:/microrna.sanger.ac.uk/sequences/ to which miRNA sequences are submitted for naming and nomenclature assignment, as well as placement of the sequences in a database for archiving and for online retrieval via the world wide web. Generally, the data collected on the sequences of miRNAs by the Sanger Institute include species, source, corresponding genomic sequences and genomic location (chromosomal coordinates), as well as full length transcription products and sequences for the mature fully processed miRNA (miRNA with a 5′ terminal phosphate group).

In one embodiment, the sample provided comprises a plurality of miRNAs of interest 60, where each of the plurality of miRNAs or other small polynucleotides of interest 60 has small polynucleotide of interest sequences that are identical to one another. In one embodiment, the sample provided comprises a plurality of miRNAs of interest 60, where at least two of the plurality of miRNAs of interest 60 have miRNA of interest sequences that are different from one another. In one embodiment, the sample provided comprises a plurality of miRNAs of interest 60 comprising a first miRNA of interest having a first miRNA of interest sequence, and a second miRNA of interest having a second miRNA of interest sequence, where the first miRNA of interest sequence is different from the second miRNA of interest sequence. In another embodiment, the sample provided comprises a plurality of miRNAs of interest 60 comprising a first miRNA of interest having a first miRNA of interest sequence, a second miRNA of interest having a second miRNA of interest sequence, and a third miRNA of interest having a third miRNA of interest sequence, where the first miRNA of interest sequence is different from the second miRNA of interest sequence, where the first miRNA of interest sequence is different from the third miRNA of interest sequence, and where second miRNA of interest sequence is different from the third miRNA of interest sequence.

Hybridization

Referring now to FIG. 1A, the method then comprises combining the capture probe 10 and the sample, represented in FIG. 1A by the small polynucleotide of interest 60. In a preferred embodiment, the method comprises combining the sample and the capture probe 10 in a solution.

In one embodiment, combining the capture probe 10 and the sample comprises combining approximately equimolar amounts of each capture probe 10. In another embodiment, combining the capture probe 10 and the sample comprises combining approximately equimolar amounts of each capture probe 10 with an amount of sample expected to contain approximately one tenth the molar amount of the small polynucleotide of interest 60 as of the capture probe 10. In another embodiment, combining the capture probe 10 and the sample comprises combining approximately equimolar amounts of each capture probe 10 with an amount of sample expected to contain approximately one half and one tenths and the molar amount of the small polynucleotide of interest as of the capture probe 10. In one embodiment, combining the capture probe 10 and the sample comprises combining the sample with between 0.1 pmoles and 100 pmoles/μl each of the capture probe 10 in a suitable buffer to create a solution comprising the capture probe 10 and the sample. In a preferred embodiment the amount of total RNA in the sample ranges from about 10 pg to about 10 μg, more preferably about 10 ng to about 1 μg. In a preferred embodiment, the buffer is selected from the group consisting of TRIS, MOPS, and SSC; includes alkali salts such as sodium chloride, lithium chloride or sodium citrate; and may further include nuclease inhibitors and accelerants such as dextran sulfate, polyethylene glycols or polyacrylamides. Exemplary buffers include, (a) 1× TE buffer in 0.1-2.0 M sodium chloride; (b) 0.1M MOPS in 1 mM EDTA and 100 mM sodium chloride, and (c) 20 mM MOPS, 1.8M Lithium Chloride, 1 mM EDTA, 100 μM aurintricarboxylic acid pH 6.8. As will be understood by those with skill in the art with reference to this disclosure, the pH selected for the buffer will be one that optimizes the intended reactions. In general, the pH selected for hybridization will be between 6 and 8, preferably between 6.4 and 7.4 and more preferably, near 7.0 (it should be noted that other buffers, buffer exchange or different buffer components from that used for hybridization may be required to afford an optimal environment compatible with the use of enzymes such as polymerases, DNases, ligases, nucleases and the like, such buffers being known in the art and the necessity for such compatibility being well understood in the art). In a preferred embodiment, the method further comprises adding one or more than one RNase inhibitor to the combination of the sample and the capture probe 10 such as for example an RNAase or nuclease inhibitor selected from the group consisting of lithium dodecylsulfate (LiDS), sodium dodecylsulfate, the ammonium salt of aurintricarboxylic acid and sodium salt of aurintricarboxylic acid, beta mercaptoethanol, dithiothreitol, Tris(2-Carboxyethyl)-Phosphine Hydrochloride (TCEP) or human placental RNase inhibitor. Such inhibitors are included to inhibit nucleases without compromising the ability of the probes and their target polynucleotides to hybridize with one another as will be understood by those skilled in the art.

Referring now to FIG. 1B, after combining the capture probe 10 and the sample, the method comprises allowing the small polynucleotide of interest 60 to hybridize with the small polynucleotide binding segment 40 to form a small polynucleotide/capture probe complex (FIG. 1B). In one embodiment, allowing the small polynucleotide of interest 60 to hybridize with the small polynucleotide binding segment 40 comprises incubating the solution comprising the capture probe 10 and the sample for between 1 minute and 60 minutes at between 25° C. and 60° C. until substantially all of the miRNA of interest 60 has hybridized to the capture probes 10, thereby sequestering the small polynucleotide of interest 10 from other substances in the sample.

Isolation/Capture

In addition to the small polynucleotide binding segment 40, some versions of the capture probes 10 also contain a solid phase binding segment 20, a spacer segment 30 and a template segment 50 capable of serving as a template for a polynucleotide polymerase. The set of capture probes 10 and hybridized polynucleotides of interest 60 can be captured to a solid phase, for example by binding of biotinylated capture probes 10 to streptavidin coated paramagnetic particles followed by temporary immobilization of the paramagnetic particles by the action of a magnet and removal of the remaining biological sample. Unlike other methods for determining small polynucleotides such as miRNAs, using the method of this disclosure permits the recovery and further processing of the removed biological sample to be analyzed for other molecular species such as mRNAs or genomic DNA This is followed by cycles of washing the particles after their release into a wash buffer to remove unhybridized polynucleotides and other materials from the paramagnetic beads and the capture-extension probe hybridization complexes.

One advantage for the immobilized capture probe 10 methods is that initial enrichment of the total RNA sample for non-protein-coding RNAs, such as small nucleolar RNAs, siRNAs, microRNAs and antisense RNAs, is not necessary. Preferably, the capture probe 10 will hybridize to the specific target in solution. Secondly, when the capture probe 10 is immobilized on the solid support, unbound material can be removed and thereby enrichment for the specific target has been performed. Another advantage is that buffer exchange can be facilitated. Yet another advantage is that at this point the small polynucleotides can be eluted from the bound capture probes. The eluted small polynucleotides are highly concentrated and enriched and are suitable for use in a wide variety of downstream analytical methods, such elution methods being well understood in the art for example use of water or formamide at 80° C., such downstream applications as gel electrophoresis, ligation and sequencing, labeling and hybridization and the like.

Extension

Next, as shown in FIG. 1B, the method comprises an extension reaction. The first step of the extension reaction comprises combining the small polynucleotide/capture probe complex with a polynucleotide polymerase and a set of nucleoside triphosphates. The extension reaction further comprises extending the hybridized small polynucleotide of interest 60 to form an extension product 80, where the extension product 80 is hybridized to the capture probe 10 to form an extension product/capture probe complex (FIG. 1C). The extension product is comprised of the small polynucleotide of interest 60 connected at the 3′ end to an extended segment comprising a sequence complementary to the template segment 50 of the capture probe 10 (FIG. 1D). In one embodiment of the invention, the extended segment 70 contains one or more labeled or modified nucleotide residues. In a preferred embodiment the extended segment contains one or more than one modified nucleotide or nucleotide analog. For example, the extended segment 70 may contain one or more internucleoside bonds, such as phosphorothioate, boranophosphate, methylphosphonate, or peptide bonds, which are nuclease resistant.

Typically, the nucleotide polymerization comprises a DNA polymerization to obtain a RNA-DNA chimera, which constitutes the extension product 80. In a preferred embodiment the DNA portion of the chimeric extension product contains a phosphorothioate backbone.

In one embodiment of the invention, the hybridized small polynucleotides 60 bound to the capture probes 10 are extended by the action of polymerase that can utilize the hybridized small RNA as a primer. In another embodiment, where the extension template segment 50 of the capture-extension probe 10 is DNA, the polymerase is a DNA dependent DNA polymerase capable of using the 3′ end of the hybridized small polynucleotide 60 as a primer. In another embodiment, the polymerase is a polynucleotide polymerase that can use RNA as primer such as T4, T7, E. coli Pol I, MMLV reverse transcriptase, Bst polymerase, Phi-29 polymerase and the like or a combination of one or more of these enzymes. In a preferred embodiment, the polynucleotide polymerase lacks any nuclease activity and can readily utilize labeled nucleoside triphosphates as substrates for its extension of the hybridized small polynucleotide, such as miRNA which serves as a primer for the extension reaction. In a particularly preferred embodiment, the polynucleotide polymerase is a thermostable DNA polymerase having an enhanced ability to incorporate modified nucleotides, such as Therminator™ DNA Polymerase, a genetically engineered variant of the native DNA polymerase from Thermococcus species 9°N-7 (New England Biolabs, Ipswich, Mass.), or Thermo Sequenase™ DNA Polymerase from Thermoplasma acidophilum (GE Healthcare Bio-Sciences Corp., Piscataway, N.J.).

The nucleotide mixture for the extension reaction comprises a set of nucleoside triphosphates, ordinarily four NTPs, e.g. ATP, CTP, GTP and UTP, or four dNTPs, e.g., dATP, dCTP, dGTP and TTP (or dUTP). In one embodiment at least one, and preferably three, of the four nucleoside triphosphates is a nucleoside triphosphate analog, such as alpha-phosphorothioate-dNTP or alpha-phosphorothioate—NTP, where a non-bridging oxygen on the alpha-phosphorus atom of the nucleoside triphosphate is replaced by a sulfur. Exemplary thiophospho deoxynucleoside triphosphates include 2′-Deoxyadenosine-5′-O-(1-Thiotriphosphate), 2′-Deoxyguanosine-5′-O-(1-Thiotriphosphate), 2′-Deoxycytidine-5′-O-(1-Thiotriphosphate), 2′-Deoxythymidine-5′-O-(1-Thiotriphosphate) and exemplary thiophospho ribonucleoside triphosphates include Adenosine-5′-O-(1-Thiotriphosphate). Cytidine-5′-O-(1-Thiotriphosphate), Guanosine-5′-O-(1-Thiotriphosphate) and Uridine-5′-O-(1-Thiotriphosphate).

In one embodiment at least one of the nucleoside triphosphates contains a detectable label such as fluorescein, cyanine 3, cyanine 5, biotin, aminoallyl, digoxigenin, tetramethylrhodamine and the like. A wide variety of detectable nucleoside triphosphates are available commercially from Roche (Indianapolis, Ind.), Invitrogen (Carlsbad, Calif.) and others. In a preferred embodiment, the labeled nucleoside triphosphate is at a lower concentration than the other three nucleoside triphosphates. For example, in one embodiment, the unlabelled nucleoside triphosphates are at a concentration in the extension reaction at between 50 and 300 micromolar and the labeled nucleoside triphosphates are at a concentration of between 5 and 30 micromolar. However, as will be understood in the art, different polymerases have different capacities to utilize such modified nucleotides in strand synthesis. Accordingly, in some cases the labeled nucleoside triphosphate may be utilized at concentrations comparable to the non-labeled nucleoside triphosphates employed in the extension reaction. Such adjustments in nucleoside triphosphate concentrations are well known in the art. Additionally, it is known in the art that the buffers and or temperatures utilized in the extension reaction can be adjusted to accommodate the incorporation of modified nucleoside triphosphates in the extension reaction.

The buffer selected for the extension reaction should not interfere with the hybridization of the small polynucleotide 60 with its capture probe 10 and be compatible with the extension reaction caused by the polymerase. Preferred versions of the buffer permit or facilitate the incorporation of modified nucleotides into the extension product.

In one embodiment the polymerase is a nuclease free form of the Klenow enzyme from E. coli, the nucleoside triphosphates are dATP, dCTP, dGTP at 100 micromolar each, the labeled dNTP is dUTP labeled with cyanine 3 at a concentration of 10 micromolar, and the extension buffer comprises 0.05M Tris-HCL, 0.01M MgCl₂, 1.0 mM DTT, 0.05 mg/ml BSA and 20 units of an RNase inhibitor such as a recombinant mammalian protein capable of inhibiting eukaryotic RNases.

In another embodiment the polymerase is a nuclease free form of a thermostable DNA polymerase from a thermophilic bacteria, like Thermococcus species 9°N-7 or Thermoplasma acidophilum, the nucleoside triphosphates are 2′-Deoxyadenosine-5′-O-(1-Thiotriphosphate), 2′-Deoxyguanosine-5′-O-(1-Thiotriphosphate) and 2′-Deoxycytidine-5′-O-(1-Thiotriphosphate), at 40 micromolar each, the labeled dNTP is dUTP labeled with cyanine 3 at a concentration of 5 nanomolar, and the extension buffer comprises 1× MOPS TBP Buffer [20 mM MOPS, 10 mM Potassium Chloride, 2 mM Magnesium Chloride, 1% Triton-X 100 with a final pH of 8.8].

Ligation

Another means to expedite labeling and/or detection of a small polynucleotide of interest is to include a ligation reaction to further lengthen the small polynucleotide extension of interest. Referring now to FIG. 2, there are shown some of the steps in certain embodiments of the methods that include a ligation reaction. The steps shown are not intended to be limiting nor are they intended to indicate that each step depicted is essential to the method, but instead are exemplary steps only.

“Ligation” or “covalent coupling” refers to covalent coupling of two adjacent nucleotide sequences, e.g. a linker sequence 100 substantially complementary to, and hybridized to, the spacer segment 30 of the capture probe covalently coupled to an adjacent miRNA or other small polynucleotide extension product 80. The reaction is catalyzed by the enzyme ligase, which forms a phosphodiester bond between the 5′-end of one nucleotide sequence and the 3′-end of the adjacent nucleotide sequence, e.g. between two adjacent segments of the capture probe or complements thereof. Suitable enzymes include the following Ligases: EC 6.5.1.1 (DNA ligase (ATP)) and EC 6.5.1.3 (RNA ligase (ATP)).

Following hybridization of the small polynucleotide of interest 60 to the capture probe 10, the method in accordance with this aspect of the present invention further comprises providing a linker segment 100 (FIG. 2A). In one embodiment, the linker segment 100 comprises a substance selected from the group consisting of one or more than one type of polynucleotide, including ribonucleotides and deoxynucleotides, one or more than one type of polynucleotide analog, and a combination of one or more than one type of polynucleotide and polynucleotide analog. In one embodiment, the linker 100 is resistant to nuclease degradation. In a preferred embodiment, the linker 100 comprises nuclease resistant nucleotides. In another preferred embodiment, the linker 100 comprises nucleotides with a phosphothioate backbone that renders the linker resistant to nuclease degradation.

The linker 100 has a linker sequence, and comprises a 3′ end and a 5′ end. In one embodiment, the linker sequence is substantially complementary to, and capable of hybridizing to, the spacer segment sequence 30 of a capture probe 10 according to the present invention by Watson-Crick base pairing.

The linker 100 comprises between 6 and 50 residues. In a preferred embodiment, the linker 100 comprises at least 10 residues, and at least 10 residues at the 3′ end of the linker 100 are exactly the complement of the corresponding residues at or near the 5′ end of the spacer segment 30.

In one embodiment, the linker 100 is allowed to hybridize to the spacer segment 30 and is then ligated to the extension product 80 to form a ligated extension product 110 substantially complementary to, and capable of hybridizing to, the capture probe sequence 10 (FIG. 2B). Such ligation reaction may be assisted by providing a linker 100 having linker sequence specific for the spacer segment sequence 30 of the capture probe 10 so that the small polynucleotide target 60 and said linker 100 are placed in close vicinity to each other upon sequence specific hybridisation.

In a preferred embodiment, the 3′ end of the linker 100 is capable of being ligated to the 5′ end of a miRNA of interest 60 by a suitable ligase, such as for example T4 polynucleotide ligase, or by another suitable chemical reaction.

Referring now to FIG. 2A, the method then comprises combining the linker 100 with the sample and the capture probe 10, represented in FIG. 2A by the small polynucleotide of interest 60 and the extended segment 70 hybridized to the capture probe 10. In a preferred embodiment, the method comprises combining the linker 100 and the hybridized capture probe/extension product 10/80 in a solution. Alternatively, the capture probe 10, the linker 100 and the sample can be combined simultaneously, or sequentially in any order, as will be understood by those with skill in the art with reference to this disclosure. For example, the capture probe 10 is combined with the sample first, and then the capture probe 10 and sample are combined with the linker 100; or alternately for example, the capture probe 10 and linker 100 are combined first, and then the capture probe 10 and linker 100 are combined with the sample; or alternately for example, the linker 100 is combined with the sample first, and then the capture probe 10 is combined with the linker 100 and the sample.

In one embodiment, combining the capture probe 10, the linker 100 and the sample comprises combining approximately equimolar amounts of the capture probe 10 and the linker 100. In another embodiment, combining the capture probe 10, the linker 100 and the sample comprises combining approximately equimolar amounts of the capture probe 10 and the linker 100 with an amount of sample expected to contain approximately one tenth the molar amount of small polynucleotide of interest 60 as of the capture probe 10 or linker 100. In one embodiment, combining the capture probe 10, the linker 100 and the sample comprises combining the sample with between 0.1 pmoles and 100 pmoles/μl each of the capture probe 10 and the linker 100 in a suitable buffer to create a solution comprising the capture probe 10, the linker 100 and the sample. In a preferred embodiment, the buffer is selected from the group consisting of TRIS, MOPS, and SSC; includes alkali salts such as sodium chloride, lithium chloride, sodium citrate; and may further include nuclease inhibitors and accelerants such as dextran sulfate, polyethylene glycols, polyacrylamides, Exemplary buffers include, (a) 1× TE buffer in 0.1-2.0 M sodium chloride; (b) 0.1M MOPS in 1 mM EDTA and 100 mM sodium chloride, and (c) 20 mM MOPS, 1.8M Lithium Chloride, 1 mM EDTA, 100 μM aurintricarboxylic acid pH 6.8. As will be understood by those with skill in the art with reference to this disclosure, the pH selected for the buffer will be one that optimizes the intended reactions. In general, the pH selected will be between 6 and 8, preferably between 6.4 and 7.4 and more preferably, near 7.0; however, the overriding consideration is that the pH as well as all buffer components be compatible with the reactions being performed and may deviate considerably from the forgoing as for example the use of buffers with a pH between 8-9 for some polymerases to afford good activity for their extension reactions Other pH values or varying buffer components may be required to afford an optimal environment compatible with the use of enzymes such as polymerases, DNases, ligases, nucleases and the like, such buffers being known in the art and the necessity for such compatibility being well understood in the art. In a preferred embodiment, the method further comprises adding one or more than one RNase inhibitor to the combination of the sample and the capture probe 10 such as for example an RNAase or nuclease inhibitor selected from the group consisting of lithium dodecylsulfate (LiDS), sodium dodecylsulfate, the ammonium salt of aurintricarboxylic acid and sodium salt of aurintricarboxylic acid, beta mercaptoethanol, dithiothreitol, Tris(2-Carboxyethyl)-Phosphine Hydrochloride (TCEP) or human placental RNase inhibitor. Such inhibitors and included to inhibit nucleases without compromising the ability of the probes and their target polynucleotides to hybridize with one another as will be understood by those skilled in the art.

Referring now to FIG. 2B, after combining the linker 100, the capture probe 10 and the sample, the method comprises allowing the linker 100 to hybridize with the spacer segment 30, thereby binding the linker 100, the small polynucleotide of interest 60, and optionally the extended segment 70 to the capture probe 10. In one embodiment, allowing the linker 100 to hybridize with the spacer segment 30 and the small polynucleotide of interest 60 to hybridize with the small polynucleotide binding segment 40 comprises incubating the solution comprising linker 100, the capture probe 10 and the sample for between 1 minute and 60 minutes at between 25° C. and 60° C. under conditions sufficient to hybridize the linker 100 to the spacer segment 30 of the capture probe 10.

In a preferred embodiment, the linker 100 hybridizes to the spacer segment 30 at a position where the last residue on the 3′ end of the linker 100 hybridizes to a residue on the spacer segment 30 that is between 1 residue and 5 residues from the 5′ end of the small polynucleotide of interest 60. In a particularly preferred embodiment, the linker 100 hybridizes to the spacer segment 30 at a position where the last residue on the 3′ end of the linker 100 hybridizes to a residue on the spacer segment 30 that is immediately adjacent to the 5′ end of the small polynucleotide of interest 60.

Next, as shown in FIG. 2B, the method comprises covalently ligating the 3′ end of the linker 100 that is hybridized to the spacer segment 30 to the 5′ end of the small polynucleotide of interest 60 that is hybridized to the small polynucleotide binding segment 40. Ligation of the 3′ end of the linker 100 to the 5′ end of the small polynucleotide of interest 60, and extension of the 3′ end of the small polynucleotide of interest to the 3′ end of the extended segment 70 can be accomplished in any order, including simultaneously or sequentially. In one embodiment, the ligation is accomplished by standard techniques, as will be understood by those with skill in the art with reference to this disclosure. In a preferred embodiment, the ligation comprises treating the capture probe 10 with the hybridized linker 100 and the extension product 80 containing a small polynucleotide of interest 60 with a suitable ligase, such as for example T4 polynucleotide ligase in the presence of suitable buffer and essential cofactors for a sufficient time for the ligation to proceed to near total completion of ligation. As will be understood by those with skill in the art with reference to this disclosure, the presence of the spacer segment 30 in the capture probe 10 facilitates the ligation of the linker 100 to the small polynucleotide of interest 60 by aligning the 3′ end of the linker 100 with the 5′ end of the small polynucleotide of interest 60. The combination of ligation and extension steps produces a “ligated extension product” 110 defined as a strand of linker 100, small polynucleotide of interest 60 and extended segment 70 that have been covalently linked together (“ligated linker-small polynucleotide of interest-extended segment”), and where the ligated extension product 110 is hybridized to the capture probe 10.

In one embodiment, the 5′ end of the linker 100 comprises a label, such as for example a fluorescent dye, to facilitate detection, as will be understood by those with skill in the art with reference to this disclosure. Further, the linker 100 can comprise a label, such as for example a fluorescent dye, to facilitate detection at a position other than at the 5′ end of the linker 100, as long as the presence of the label does not interfere with other steps of the present method, as will be understood by those with skill in the art with reference to this disclosure. In other embodiments, the linker sequence 100 joined to the small polynucleotide of interest 60 by ligation may accommodate in part primers for PCR amplification or for a labeled detection probe, alone or in combination with the nucleic acid sequence of the adjacent small polynucleotide 60.

Capture Probe Degradation

Next, as shown in FIG. 1D or FIG. 2C, certain embodiments of the method of the present invention include the step of degrading the capture probe, but not the extension product, to obtain a single stranded extension product 80 (FIG. 1D) or ligated extension product 110 (FIG. 2C).

In one embodiment the capture probe 10 can be degraded by a nuclease or combination of nucleases, especially DNase I and the like, while a chimeric extension product 80 (FIG. 1D) or ligated extension product 110 (FIG. 2C) will not be degraded by the action of the nuclease. For example, the capture probe can be comprised of a DNA polynucleotide with ordinary phosphate linkages in its back bone. Upon hybridization with the polynucleotide of interest, e.g., a microRNA, the polynucleotide of interest is then extended by the action of a suitable DNA polymerase using alpha thio deoxynucleoside triphosphates. A double stranded species will result, where one strand is the DNA capture probe 10 and the other strand is the chimeric extension product 80 comprised of an extended micro RNA with a DNA extension segment having a phosphorothioate backbone. Those skilled in the art will recognize that the capture probe can be degraded by treatment with DNase I leaving the chimeric extension product 80 comprised of the target microRNA, i.e., the polynucleotide of interest 60, and its DNA extended segment 70 unaffected and single stranded.

Accordingly, in one embodiment the extension product 80 produced by the extension reaction is a chimeric polynucleotide comprised of the small polynucleotide of interest 60 connected at the 3′ end to an extended segment 70, which is resistant to cleavage by the nuclease, and the capture probe is degraded by a nuclease, such as DNase I. In a preferred embodiment the polynucleotide of interest 60 is a small RNA, such as an miRNA, snoRNA, siRNA or short interfering RNA, and the extended segment 70 contains a phosphorothioate backbone. In a particularly preferred embodiment, the extended segment 70 is a phosphorothiolated DNA polynucleotide.

In another embodiment the ligated extension product 110 is comprised of a linker 100, a small polynucleotide of interest 60 and an extended segment 70, which are resistant to cleavage by the nuclease, and the capture probe is degraded by a nuclease, such as DNase I. In a preferred embodiment the polynucleotide of interest 60 is a small RNA, such as an miRNA, snoRNA, siRNA or short interfering RNA, and both the linker 100 and the extended segment 70 have a phosphorothioate backbone. In a particularly preferred embodiment, the linker 100 and the extended segment 70 are both phosphorothiolated DNA polynucleotides.

Alternatively the capture probe can be degraded by chemical decomposition. For example, the capture probe 10 can be comprised of a polynucleotide with phosphorothioate linkages in its back bone. Upon hybridization with the polynucleotide of interest 60, for example where it is a microRNA, the polynucleotide of interest is then extended by the action of a suitable DNA polymerase. A double stranded species will result where one strand is the phosphorothiolated capture probe 10 and the opposite strand is the extended micro RNA, i.e., the polynucleotide of interest 60 with a DNA extension segment 70. In this example, those skilled in the art will recognize that the capture probe 10 can be degraded by treatment with iodine leaving the chimeric extension product 80 comprised of the microRNA and its DNA extended segment unaffected and single stranded.

Accordingly, in an alternative embodiment the capture probe 10 is a polynucleotide with phosphorothioate linkages, whereas the extension product 80 or ligated extension product 110 contains ordinary phosphate linkages, and the capture probe 10, but not the extension products, is degraded by treatment with iodine. In a preferred embodiment, the capture probe 10 is a phosphorothiolated DNA polynucleotide. In one version the extension product 80 is a chimeric polynucleotide where the polynucleotide of interest 60 is a small RNA, such as an miRNA, snoRNA, siRNA or short interfering RNA, and the extended segment is a DNA polynucleotide. In another version, the polynucleotide of interest is a small DNA and the extended segment is a DNA polynucleotide.

### Detection/Identification

Analysis of extension products 80 can be performed using techniques known in the art including, without limitation, hybridization and detection by the use of a microarray specific for the miRNAs or other small polynucleotides to be evaluated, polymerase chain reaction (PCR)-based analysis, sequence analysis, flow cytometry and electrophoretic analysis.

Reverse Transcription

Subsequent amplification, detection, and/or identification of the polynucleotide of interest 60 in many embodiments may further comprise reverse transcription of the resulting extension product 80 to produce cDNA. The design of suitable reverse transcription primers and use of reverse transcriptase to produce cDNA copies of extension products may can be accomplished by any means known to one of skill in the art with reference to the present disclosure.

Amplification

The terms “PCR reaction”, “PCR amplification”, “PCR”, “pre-PCR” and “real-time quantitative PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence based amplification (NASBA™, Cangene, Mississauga, Ontario) and Q Beta Replicase systems. The products formed by said amplification reaction may or may not be monitored in real time or only after the reaction as an end point measurement.

### Kits

In another embodiment of the present invention, there is provided a kit containing one or reagents for use in the isolation, labeling, and/or detection of small RNAs, such as for example, human miRNAs. Preferred versions of the kits can include, (a) an equimolar mix of capture probes; (b) a nucleotide mix containing deoxyribonucleoside triphosphates or ribonucleoside triphosphates; (c) a polymerase, (d) streptavidin coated paramagnetic beads; (e) a ligase enzyme; (g) an oligonucleotide linker that is substantially complementary to and capable of hybridizing to the spacer segment of the capture probes; or (h) one or more than restriction enzyme specific for a restriction enzyme recognition sequence contained in the capture probes.

In one embodiment, the kit comprises an equimolar mix of capture extension probes according to the present invention, such as SEQ ID NOs 9 and 10 set forth in the attached sequence listing and listed in Table II of the examples, with the small RNA binding segments comprising complementary sequences to the known human mature miRNA population. In one embodiment, the kit further comprises one or more than one substance selected from the group consisting of labeling buffer comprising 0.5M Tris-HCL, 0.1M MgCl₂ 10 mM DTT, 0.5 mg/ml BSA and an RNase inhibitor, such as a recombinant mammalian protein capable of inhibiting eukaryotic RNases; a nucleotide mix containing for example Cyanine 3-dUTP or Cyanine 5-dUTP at 10 micromolar each and unlabeled dATP, dCTP and dGTP at 100 micromolar each; a labeling enzyme such as a polynuclease polymerase for example, Exonuclease-Free Klenow (USB Corp.; Cleveland, Ohio US); capture beads such as 1 micron streptavidin coated paramagnetic beads; bead wash buffer comprising for example 0.5M Tris-HCL, 0.1M MgCl₂, and 10 mM DTT; labeled miRNA elution buffer comprising for example formamide; a buffer exchange device such as Microcon® YM 10 devices and 0.1× TE wash buffer.

In a particularly preferred embodiment a kit is provided with one or more capture probes capable of binding or hybridization with one or more small polynucleotides of interest, a deoxynucleotide triphosphate mix consisting of the alpha thio triphosphate forms of dATP, dCTP, and dGTP and a labeled form of dUTP, a polymerase capable of extending the 3′ end of the targeted polynucleotides hybridized to the capture probes and using the extension template segment of the capture probe as a template for extension, suitable buffers compatible with the polymerase, DNase I and/or Alkaline phosphatase reactions, spin columns for separation of unincorporated nucleoside triphosphates from the labeled extension products containing the polynucleotide of interest and instructions for the use of the kit.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features.

The foregoing discussion is by no means limiting and other means of detecting the miRNAs labeled and measured by the method of the present invention can readily be envisioned such as the detection of the extended population of miRNAs by electrophoresis, or for example by the extension of the miRNAs where the sample itself serves as the solid phase such as tissue sections. The invention is described in more detail by the following description.

### EXAMPLE 1

### Labeled Extension with Probe Digestion Example

One embodiment of the present method was performed as follows.

Synthetic miRNA

The synthetic miRNA(syn-miRNA) were selected and designed to specifically reflect a set of human miRNA. The syn-miRNA were obtained from Integrated DNA Technologies, (Coralville, Iowa). The syn-miRNA were resuspended a stabilization buffer containing 1 mM Sodium Citrate (Ambion; Austin, Tex.) and 30% Formamide (Bioventures; Murfreesboro, Tenn.) to a final concentration of 100 pmol/μl. The syn-miRNA's were then aliquoted into 10 μl working stocks in 0.5 ml tubes (Nalgene; Rochester, N.Y.) to reduce freeze-thaw effects.

TABLE 1

| | | |
|---|---|---|
| NAME | SEQUENCE | SEQ ID NO |

| | | |
|---|---|---|
| hsa-let-7a | /5Phos/rUrGrArGrGrUrArGrUr | SEQ ID NO:1 |
| | ArGrGrUrUrGrUrArUrArGrUrU | |
| hsa-let-7e | /5Phos/rUrGrArGrGrUrArGrGr | SEQ ID NO:2 |
| | ArGrGrUrUrGrUrArUrArGrU | |
| hsa-miR-106a | /5Phos/rArArArArGrUrGrCrUr | SEQ ID NO:3 |
| | UrArCrArGrUrGrCrArGrGrUrAr | |
| | GrC | |
| hsa-miR-126* | /5Phos/rCrArUrUrArUrUrArCr | SEQ ID NO:4 |
| | UrUrUrUrGrGrUrArCrGrCrG/ | |
| hsa-miR-135a | /5Phos/rUrArUrGrGrCrUrUrUr | SEQ ID NO :5 |
| | UrUrArUrUrCrCrUrArUrGrUrGr | |
| | A | |
| hsa-miR-138 | /5Phos/rArGrCrUrGrGrUrGrUr | SEQ ID NO:6 |
| | UrGrUrGrArArUrC | |
| hsa-miR-154 | /5Phos/rUrArGrGrUrUrArUrCr | SEQ ID NO:7 |
| | CrGrUrGrUrUrGrCrCrUrUrCrG | |
| hsa-miR-154* | /SPhos/rArArUrCrArUrArCrAr | SEQ ID NO:8 |
| | CrGrGrUrUrGrArCrCrUrArUrU | |

### ILLUMINATE Capture Probes

The capture probes were selected and designed to be specifically complementary to the human miRNA's with the addition of a spacer segment and a template segment. The miRNA capture probe complexes were obtained from Integrated DNA Technologies, (Coralville, Iowa). The ILLUMINATE capture probes were resuspended in 0.1×TE buffer with 2% Acetonitrile (Sigma Aldrich; St. Louis, Mo.) for a final concentration of each probe of 100 pmol/μl.

TABLE 2

| ILLUMINATE | PROBE | | |
|---|---|---|---|
| CAPTURE | SEQUENCE | COMPLEMENTARY | |
| PROBE NAME | 5′-3′ | miRNA | SEQ ID NO |
| 38-ILLUM- | TAATACGACTCAC | hsa-miR-138 | SEQ ID NO:9 |
| HSA138 | TATAGGGGATTCA | | |
| | CAACACCAGCTTC | | |
| | TTCACA | | |
| 13-ILLUM- | TAATACGACTCAC | hsa-miR-106a | SEQ ID NO:10 |
| HSA-106a | TATAGGGGCTACC | | |
| | TGCACTGTAAGCA | | |
| | CTTTTTCTT | | |

Hybridization and Labeling of microRNA to ILLUMINATE Capture Probe

Hybridization was carried out by adding 25 pmol of 13-ILLUM-HSA-106a capture probe into a well of a Bio-Rad 96-well Multiplate, 20 pmol of synthetic miRNA, hsa-miR-106a, and 1× MOPS TBP Buffer, 20 mM MOPS (Sigma Chemical Co; St. Louis, Mo.), 10 mM Potassium Chloride (American Bioanalytical; Natick, Mass.), 2 mM Magnesium Chloride (Sigma Chemical Co; St. Louis, Mo.), 1% Triton-X 100 (Calbiochem; San Diego, Calif.) with a final pH of 8.8) to a final volume of 18.5 μl. The plate was then briefly pulsed in a centrifuge to mix components and placed on a thermocycler (Bio-Rad; Hercules, Calif.) block control and a heated lid with a program consisting of 60° C. for 1 minute, followed by a decrease in temperature from 60° C. to 23° C. at an interval of 1° C. per second. Following hybridization the following was added to the wells to complete the microRNA labeling, 40 μm each 2′-dCsTP, 2′-dGsTP and 2′-dAsTP (USB Corp.; Cleveland, Ohio), 5 nmol Cyanine3 dUTP (Enzo Life Sciences, Inc.; Farmingdale, N.Y.) and 50 mM Dithiothreitol (Sigma Chemical Co) and 5 units of Therminator™ DNA polymerase (NEB; Ipswich, Mass.). The plate was then briefly pulsed in a centrifuge to mix components and placed on a thermocycler with block control and a heated lid with a program consisting of 40° C. for 1 minute, followed by a 0.1° C. per second increase to 60° C., then 60° C. for 20 minutes.

### Cleavage of the Capture Probe and Reaction Clean-up

After the extension and labeling of the microRNAs a mixture of 1 unit of Shrimp Alkaline Phosphatase and 1 unit of DNaseI (Promega Corp.; Madison, Wis.) was added to each well to cleave nonspecifically the DNA capture probe to release di-, tri- and oligonucleotide products and to remove the 5′ and 3′phosphate group from the nucleotides. The DNase enzyme does not recognize RNA or thio-dNTPs resulting in only the capture probe being cleaved into small oligonucleotide products. The plate was then briefly pulsed in a centrifuge to mix components and placed on a thermocycler with block control and a heated lid at 37° C. for 10 minutes. The enzymes were inactivated by the addition of 1 μl 0.1% sodium dodecyl sulfate (VWR; West Chester, Pa.)

Micro Select-D, G25 TE (IBI-Shelton SCIENTIFIC; Peosta, Iowa) were used to remove the cleaved capture probe products and unincorporated dNTPs. The Micro Select-D columns were placed in a 2 ml collection tubes (IBI-Shelton SCIENTIFIC). The column/collection tubes were placed in a microcentrifuge, IEC MicromaxRX (Thermo Scientific; Waltham, Mass.) and spun at 1,000×g for 5 minutes to remove the hydrating fluid. The columns were then placed into a new 2 ml collection tubes and the sample was loaded onto the column being careful not to disturb the column resin. After 3 minutes the column/collection tubes were placed in the microcentrifuge and spun at 1,000×g for 5 minutes to collect the purified labeled microRNA.

Analysis of the microRNA Labeling

1 μl of the cleaned reaction product was added to a new 96-well Multiplate (Bio-Rad) containing 18.5 μl DI Formamide (BioVentures) and 0.5 μl of 20 fmol each of Hex labeled oligonucleotides ranging in size from 15 bp, 20 bp, 25 bp, 30 bp, 35 bp, 40 bp, 45 bp, 50 bp. The plate was then briefly pulsed in a centrifuge, Allegra 31 (Beckman Coulter; Fullerton, Calif.) to mix components then placed on an ABI Prism® 3100 DNA Analyzer (Applied Biosystems; Foster City, Calif.) using the Genescan program, Dye Set “D,” module file “GeneScan_{—}030507_microshort,” with an injection voltage of 1 kvolt, injection time of 22 seconds and a run times of 1000 seconds. The raw data was analyzed using GeneScan software (Applied Biosystems).

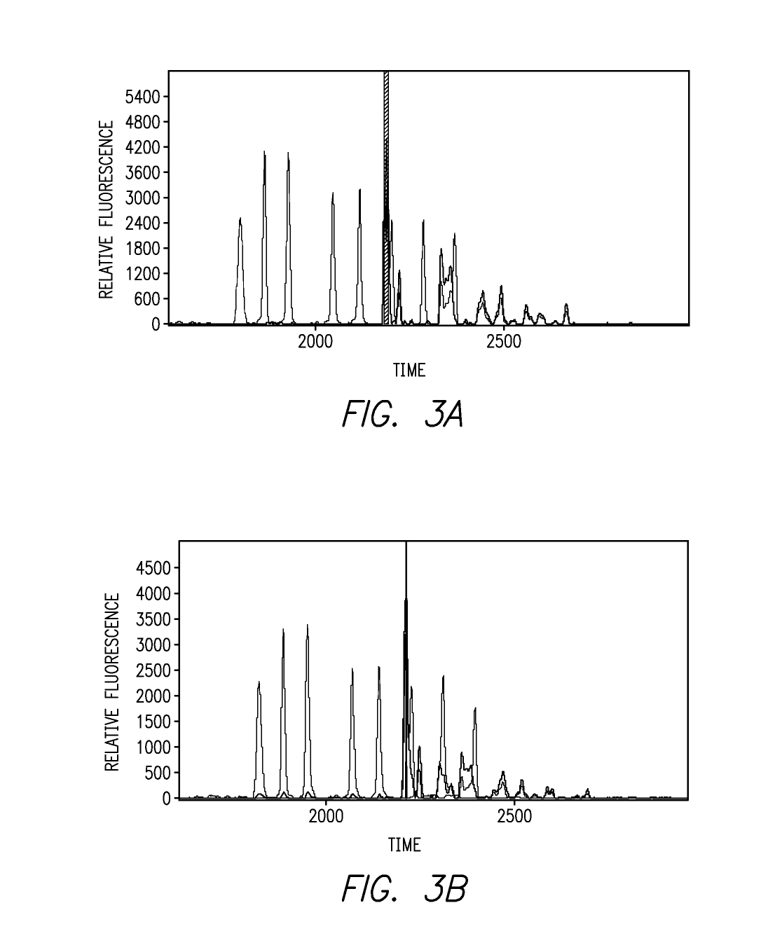

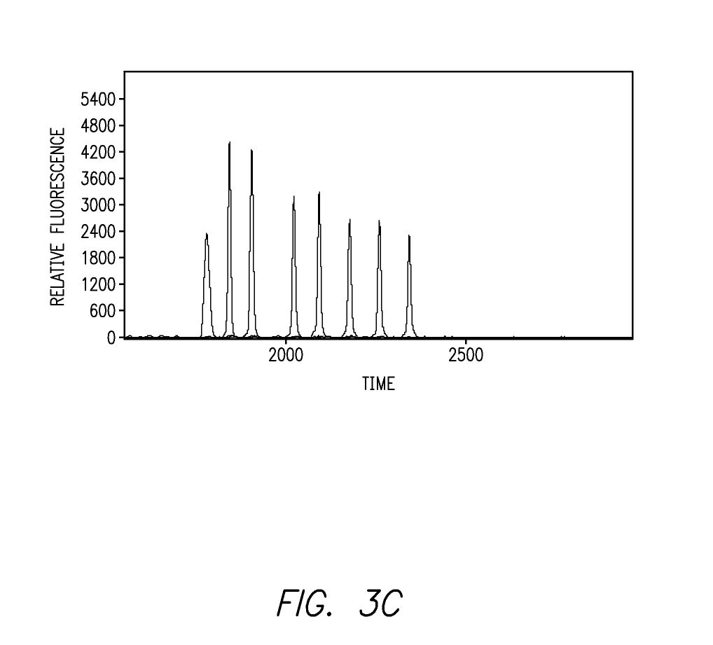

FIGS. 3A to 3C demonstrate that the polymerase extends when a microRNA is hybridized to its complementary capture probe. The extension reaction incorporated Cyanine 3 labeled dUTPs, resulting in a labeled oligonucleotide that is 42 base pairs in length, comprising the microRNA of interest and the complement to the template segment of the capture probe as shown in FIG. 3A. After the extension reaction, the DNase enzyme cleaves only the capture probe into small di- or tri-oligonucleotide products, which are then removed during the G25 column cleanup as shown in FIG. 3B. The labeled extension product is resistant to the DNase action by the use of alpha thio-dNTP's. FIG. 3B shows that the extension product does not fragment after the DNase treatment. FIG. 3C shows if there is not a complementary microRNA in the reaction, no labeled product will result.

The present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

What is claimed is:

1. A method of labeling and/or detecting a small polynucleotide of interest, comprising:
(a) providing one or more than one capture probe, wherein the capture probe is a polynucleotide comprising;
(1) a small polynucleotide binding segment having a small polynucleotide binding segment sequence, the small polynucleotide binding segment having a 3′ end and a 5′ end; and
(2) a template segment having an template segment sequence, the template segment having a 3′ end and a 5′ end;
where the small polynucleotide binding segment is substantially complementary to, and capable of hybridizing to, one or more than one small polynucleotide of interest by Watson-Crick base pairing, where the small polynucleotide of interest is selected from the group consisting of a RNA polynucleotide, a DNA polynucleotide and a combination thereof; and
where the 3′ end of template segment is connected to the 5′ end of the small polynucleotide binding segment;
(b) providing a sample comprising a small polynucleotide of interest, where the small polynucleotide of interest is selected from the group consisting of a RNA polynucleotide, a DNA polynucleotide and a combination thereof;
(c) combining the capture probe and the sample;
(d) allowing the small polynucleotide of interest to hybridize with the small polynucleotide binding segment of the capture probe to form a small polynucleotide/capture probe complex;
(e) combining the small polynucleotide/capture probe complex with a polynucleotide polymerase and a set of nucleoside triphosphates;
(f) extending the hybridized small polynucleotide of interest to form an extension product, the extension product comprising the small polynucleotide of interest connected at the 3′ end to an extended segment, the extended segment sequence comprising a sequence complementary to the template segment sequence of the capture probe, where the extension product is hybridized to the capture probe to form an extension product/capture probe complex; and
(g) degrading the capture probe to obtain a single stranded extension product.

2. The method of claim 1 wherein the small polynucleotide of interest is selected from the group consisting of miRNAs, snoRNAs, siRNAs or short interfering RNAs.

3. The method of claim 1, wherein the small polynucleotide binding segment is substantially complementary to, and capable of hybridizing to a miRNA of interest.

4. The method of claim 1, the capture probe further comprising a spacer segment having a spacer segment sequence, the spacer segment having a 3′ end and a 5′ end, where the 5′ end of the spacer segment is connected to the 3′ end of the small polynucleotide binding segment.

5. The method of claim 1, wherein the capture probe also contains a solid phase binding segment of a molecular composition capable of binding to a solid phase.

6. The method of claim 5, wherein the small polynucleotide/capture probe complex or the extension product/capture probe complex is captured to a solid phase by binding of the capture probe to a solid support via the solid phase binding segment.

7. The method of claim 1, wherein the one or more than one capture probe is a composition comprising two or more capture probes, the composition comprising:
(a) a first capture probe having a first small polynucleotide binding segment and a first template segment; and
(b) a second capture probe having a second small polynucleotide binding segment and a second template segment, where the second small polynucleotide binding segment has a different polynucleotide binding segment sequence than the first polynucleotide binding segment and the second template segment has a different template segment sequence than the first template segment.

8. The method of claim 1, wherein one or more than one of the nucleoside triphosphate contains a detectable label.

9. The method of claim 1 wherein at least three of the nucleoside triphosphates are nucleoside triphosphate analogs, where an alpha-phosphorus atom of the nucleoside triphosphate is replaced by sulfur.

10. The method of claim 1, wherein at least three of the nucleotide triphosphates are selected from the group consisting of 2′-Deoxyadenosine-5′-O-(1-Thiotriphosphate), 2′-Deoxyguanosine-5′-O-(1-Thiotriphosphate), 2′-Deoxycytidine-5′-O-(1-Thiotriphosphate), and 2′-Deoxythymidine-5′-O-(1-Thiotriphosphate).

11. The method of claim 2 wherein the extension product is a chimeric polynucleotide, wherein the extended segment is a DNA polynucleotide.

12. The method of claim 2 wherein the extended segment contains a phosphorothioate backbone.

13. The method of claim 12, the degrading step includes treating the extension product/capture probe complex with one or more than one nuclease.

14. The method of claim 13, wherein the nuclease is DNase I.

15. The method of claim 1, wherein the capture probe is a phosphorothiolated polynucleotide and the degrading step comprises treating the extension product/capture probe complex with iodine.

16. A method for labeling and/or detecting a small polynucleotide of interest in a sample comprising:
(a) providing one or more than one capture probe, the capture probe comprising
(1) a spacer segment having a spacer segment sequence, the spacer segment having a 3′ end and a 5′ end.
(2) a small polynucleotide binding segment having a small polynucleotide binding segment sequence, the small polynucleotide binding segment having a 3′ end and a 5′ end; and
(3) a template segment having an template segment sequence, the template segment having a 3′ end and a 5′ end;
where the 5′ end of the spacer segment is connected to the 3′ end of the small polynucleotide binding segment;
where the small polynucleotide binding segment is substantially complementary to, and capable of hybridizing to, one or more than one small polynucleotide of interest by Watson-Crick base pairing, where the small polynucleotide of interest is selected from the group consisting of a RNA polynucleotide, a DNA polynucleotide and a combination thereof; and
where the 3′ end of template segment is connected to the 5′ end of the small polynucleotide binding segment.
(b) providing a sample comprising a small polynucleotide of interest, where the small polynucleotide of interest is selected from the group consisting of a RNA polynucleotide, a DNA polynucleotide and a combination thereof;
(c) combining the capture probe and the sample;
(d) allowing the small polynucleotide of interest to hybridize with the small polynucleotide binding segment of the capture probe to form a small polynucleotide/capture probe complex;
(e) combining the small polynucleotide/capture probe complex with a polynucleotide polymerase and a set of nucleoside triphosphates;
(f) extending the hybridized small polynucleotide of interest to form an extension product, the extension product comprising the small polynucleotide of interest connected at the 3′ end to an extended segment, the extended sequence comprising a sequence complementary to the template segment of the capture probe, where the extension product is hybridized to the capture probe to form a extension product/capture probe complex; and
(g) providing a ligase and a linker segment, the linker segment comprising a polynucleotide having 3′ end and a 5′ end, the linker segment having a linker segment sequence, wherein the linker segment sequence is substantially complementary to, and capable of hybridizing to, the spacer segment sequence by Watson-Crick base pairing;
(h) allowing the linker segment to hybridize to the spacer segment;
(i) ligating the 3′ end of the linker segment to the 5′ end of the small polynucleotide of interest to form a ligated extension product substantially complementary to, and capable of hybridizing to, the capture probe sequence; and
(j) degrading the capture probe by nuclease treatment to obtain a single stranded ligated extension product.

17. The method of claim 16 wherein the small polynucleotide of interest is selected from the group consisting of miRNAs, snoRNAs, siRNAs or short interfering RNAs.

18. The method of claim 16, wherein the small polynucleotide binding segment is substantially complementary to, and capable of hybridizing to a miRNA of interest.

19. The method of claim 16, wherein the capture probe also contains a solid phase binding segment of a molecular composition capable of binding to a solid phase.

20. The method of claim 19, wherein the small polynucleotide/capture probe complex or the extension product/capture probe complex is captured to a solid phase by binding of capture probe to a solid support via the solid phase binding segment.

21. The method of claim 16, wherein the one or more than one capture probe is a composition comprising two or more capture probes, the composition comprising:
(a) a first capture probe having a first spacer segment, a first small polynucleotide binding segment and a first template segment; and
(b) a second capture probe having a second spacer segment, a second small polynucleotide binding segment and a second template segment, where the second small polynucleotide binding segment has a different polynucleotide binding segment sequence than the first polynucleotide binding segment and the second template segment has a different template segment sequence than the first template segment.

22. The method of claim 16, wherein one or more than one of the nucleoside triphosphate contains a detectable label.

23. The method of claim 16 wherein at least three of the nucleoside triphosphates are nucleoside triphosphate analogs, where an alpha-phosphorus atom of the nucleoside triphosphate is replaced by sulfur.

24. The method of claim 16, wherein at least three of the nucleotide triphosphates are selected from the group consisting of 2′-Deoxyadenosine-5′-O-(1-Thiotriphosphate), 2′-Deoxyguanosine-5′-O-(1-Thiotriphosphate), 2′-Deoxycytidine-5′-O-(1-Thiotriphosphate), and 2′-Deoxythymidine-5′-O-(1-Thiotriphosphate).

25. The method of claim 17 wherein the ligated extension product is a chimeric polynucleotide, wherein the linker segment and the extended segment are DNA polynucleotides.

26. The method of claim 17 wherein linker segment and the extended segment both contain a phosphorothioate backbone.

27. The method of claim 26, wherein the degrading step includes treating the ligated extension product/capture probe complex with one or more than one nuclease.

28. The method of claim 27, wherein the nuclease is DNase I.

29. The method of claim 16, wherein the capture probe is a phosphorothiolated polynucleotide and the degrading step comprises treating the ligated extension product/capture probe complex with iodine.

30. The method of claim 16, wherein the linker segment or the extended segment contains a detectable label.

31. The method of claim 16, further comprising amplifying the ligated extension product by a polymerase chain reaction.

32. A kit for the isolation, labeling and/or detection of small polynucleotides, the kit comprising:
(a) one or more capture probes comprising
(1) a small polynucleotide binding segment having a small polynucleotide binding segment sequence, the small polynucleotide binding segment having a 3′ end and a 5′ end; and
(2) a template segment having an template segment sequence, the template segment having a 3′ end and a 5′ end;
where the small polynucleotide binding segment is substantially complementary to, and capable of hybridizing to, one or more than one small polynucleotide of interest by Watson-Crick base pairing, where the small polynucleotide of interest is selected from the group consisting of a RNA polynucleotide, a DNA polynucleotide and a combination thereof; and
where the 3′ end of template segment is connected to the 5′ end of the small polynucleotide binding segment;
(3) an optional spacer segment having a spacer segment sequence, the spacer segment having a 3′ end and a 5′ end, where the 5′ end of the spacer segment is connected to the 3′ end of the small polynucleotide binding segment; and
(4) an optional solid phase binding segment containing biotin; and
(b) one or more than one substance selected from the group consisting of:
(1) a deoxynucleoside triphosphate mix comprising the alpha thio triphosphate forms of dATP, dCTP, and dGTP and a labeled form of dUTP;
(2) a polymerase capable of extending the 3′ end of a polynucleotide of interest hybridized to the capture probe by using the extension template segment of the capture probe as a template for an extension reaction;
(3) an oligonucleotide linker that is substantially complementary to and capable of hybridizing to the spacer segment of the capture probe;
(4) a ligase enzyme;
(5) DNase I;
(6) alkaline phosphatase;
(7) suitable buffers compatible with one or more of the polymerase, ligase, DNase I and alkaline phosphatase reactions;
(8) spin columns for separation of unincorporated nucleoside triphosphates from labeled extension products; and
(9) streptavidin coated paramagnetic beads.
